(19) United States
(12) Patent Application Publication
(10) Pub. No.: US 2017/0022578 A1
(43) Pub. Date: Jan. 26, 2017

(54) DEVICE AND METHOD FOR PRODUCING ELECTRIC ENERGY FOR PRODUCING IRON FROM OXIDE IRON ORES

(71) Applicant: EVONIK DEGUSSA GMBH, Essen (DE)

(72) Inventors: GEORG MARKOWZ, Alzenau (DE); RAOUL NEUHAUS, Rodenbach (DE); FLORIAN BÖSS, Hanau (DE); HARALD SCHNEIDER, Marl (DE); MARTIN MÖLLER, Dülmen (DE)

(73) Assignee: EVONIK DEGUSSA GMBH, Essen (DE)

(21) Appl. No.: 15/300,975

(22) PCT Filed: Mar. 20, 2015
(86) PCT No.: PCT/EP2015/055934

§ 371 Date: Sep. 30, 2016

(30) Foreign Application Priority Data
Apr. 3, 2014 (DE) 10 2014 206 423.6

(51) Int. Cl. C21B 13/00; C07C 2/76; C21B 5/00

(52) U.S. Cl. CPC C21B 13/004; C21B 5/001; C21B 13/0073; C07C 2/76; C21B 2005/005

(57) ABSTRACT

In an apparatus, comprising a plant for the electrothermic preparation of ethyne from coal or a hydrocarbon-containing gas providing a hydrogen-containing gas, a plant for the production of iron by reduction of oxidic iron ores, and at least one gas conduit which feeds the hydrogen-containing gas from the plant for the electrothermic preparation of ethyne as reducing agent to the plant for the production of iron by reduction of oxidic iron ores, electric energy can be utilized for the production of iron from oxidic iron ores by feeding a hydrogen-containing gas, obtained in the electrothermic preparation of ethyne from coal or a hydrocarbon-containing gas, as reducing agent to the plant for the production of iron by reduction of oxidic iron ores.

The invention relates to an apparatus and a process which allow using electric energy for producing iron from oxidic iron ores.

The production of iron from oxidic iron ores is usually carried out in a blast furnace process by reduction of oxidic iron ore with carbon monoxide produced by air oxidation of coke. Large amounts of carbon dioxide are formed here. The production of iron in a blast furnace process therefore leads to large carbon dioxide emissions from fossil carbon sources.

To reduce the emissions of carbon dioxide from the blast furnace process, hydrocarbons, such as mineral oil or natural gas, or molecular hydrogen can be fed as reducing agent to the blast furnace as replacement for part of the coke.

As alternatives to the blast furnace process, a series of direct reduction processes have been developed, in which reduction is carried out with carbon monoxide-containing synthesis gas or another reducing agent instead of the reduction with carbon monoxide produced from coke. However, these processes also generally utilize fossil carbon sources as reducing agents or as starting material for the production of the reducing agent.

To reduce the carbon dioxide emissions further, it is desirable to be able to use energy from renewable sources, in particular from solar energy or wind power, instead of fossil energy sources for producing iron from oxidic iron ores. However, these renewable sources provide energy in the form of electric energy which cannot be used directly for the reduction of oxidic iron ore in the blast furnace process and the direct reduction processes.

To be able to utilize electric energy produced from renewable sources in the blast furnace process, it has been proposed that hydrogen be produced by electrolysis of water and the hydrogen be fed as reducing agent to the blast furnace. However, such an electrolysis is technically complicated and uneconomical.

For this reason, there is still a need to be able to utilize electric energy more efficiently for the production of iron from oxidic iron ores in order to reduce the emissions of carbon dioxide in iron production. In particular, there is a need for processes which allow better use of electric energy than the use of hydrogen produced electrolytically from water as reducing agent.

It has now been found that this can be achieved by coupling an electrothermic preparation of ethyne with production of iron from oxidic iron ores, in which hydrogen from the electrothermic preparation of ethyne is used as reducing agent for the production of iron from oxidic iron ores.

The invention accordingly provides an apparatus for utilizing electric energy for the production of iron from oxidic iron ores, which comprises a plant for the electrothermic preparation of ethyne from coal or a hydrocarbon-containing gas providing a hydrogen-containing gas, a plant for the production of iron by reduction of oxidic iron ores, and at least one gas conduit which feeds the hydrogen-containing gas from the plant for the electrothermic preparation of ethyne as reducing agent to the plant for the production of iron by reduction of oxidic iron ores.

In addition, the invention provides a process for utilizing electric energy for the production of iron from oxidic iron ores, which comprises an electrothermic preparation of ethyne from coal or a hydrocarbon-containing gas providing a hydrogen-containing gas and introduction of this gas as reducing agent into a plant for the production of iron by reduction of oxidic iron ores.

The apparatus of the invention comprises a plant for the electrothermic preparation of ethyne from coal or a hydrocarbon-containing gas providing a hydrogen-containing gas.

In an electrothermic preparation of ethyne, ethyne is prepared in an endothermic reaction from hydrocarbons or coal, and the heat required for carrying out the reaction is generated by electrical power. It is possible to use gaseous or vaporized hydrocarbons, preferably aliphatic hydrocarbons, for the electrothermic preparation of ethyne. Particularly suitable are methane, ethane, propane and butanes, more particularly methane. Suitable plants for the electrothermic preparation of ethyne are known from the prior art, for example from Ullmann's Encyclopedia of Industrial Chemistry, 5th Edition, Vol. A1, pages 115-122, from DE 1 900 644 A1, from EP 0 133 982 A2 and from H. Brachold et al., Chem.-Ing.-Tech. 65 (1993), pages 293-297.

In the electrothermic preparation of ethyne, hydrogen is obtained as coproduct in addition to ethyne. The reaction mixture obtained in the electrothermic preparation of ethyne is therefore a gas containing ethyne and hydrogen which can additionally contain soot and hydrocarbons other than ethyne.

The plant for the electrothermic preparation of ethyne can comprise one or more apparatuses in which ethyne is produced electrothermically. If the plant comprises a plurality of apparatuses for the production of ethyne, these are preferably arranged in parallel and can be operated independently of one another. The use of a plurality of apparatuses arranged in parallel makes it possible, by the switching on and off of individual apparatuses, to alter the generation of ethyne in stages, while maintaining the optimum operating conditions in the individual apparatuses, and prevents efficiency losses resulting from partial load operation.

The plant for the electrothermic preparation of ethyne preferably comprises an electric arc reactor. The electrothermic preparation of ethyne may in this case take place in a one-stage process, in which at least one hydrocarbon is passed through the arc with a gas stream. Alternatively, the electrothermic preparation of ethyne may take place in a two-stage process, in which hydrogen is passed through the arc and at least one hydrocarbon is fed downstream of the arc into the hydrogen plasma produced in the arc. The plant for the electrothermic preparation of ethyne preferably comprises a plurality of electric arc reactors which are arranged in parallel and can be operated independently of one another.

The plant for the electrothermic preparation of ethyne preferably comprises an apparatus which allows rapid cooling (quenching) of all or part of the reaction mixture obtained in the electrothermic preparation of ethyne. The reaction mixture is preferably cooled to temperatures of less than 250° C. The rapid cooling may be accomplished using a direct quenching method such as, for example, the introduction of hydrocarbons and/or water, or an indirect quenching method, such as, for example, rapid cooling in a heat exchanger with generation of steam. Direct quenching and indirect quenching may also be combined with one another. In a first embodiment, the reaction mixture is quenched only with water. This embodiment features relatively low capital costs. In a preferred embodiment, the reaction mixture is mixed with a hydrocarbon-containing gas or a hydrocarbon-containing liquid, with at least part of the hydrocarbons being endothermally cracked. Depending on the process regime, a more or less broad product spectrum is produced, including for example, in addition to ethyne, hydrogen and possibly carbon monoxide, fractions of ethane, propane, ethene and other lower hydrocarbons. As a result, the heat produced can be put to a further use, such as the endothermic cracking of hydrocarbons, to a substantially greater extent. Suitable apparatuses for quenching the reaction mixture are known from the prior art, for example from Ullmann's Encyclopedia of Industrial Chemistry, 5th Edition, Vol. A1, pages 108-110 and 116-118.

The plant for the electrothermic preparation of ethyne preferably comprises an apparatus for separating soot from the reaction mixture obtained in the electrothermic preparation of ethyne to provide a product gas. This product gas contains the ethyne produced, the hydrogen formed as coproduct and generally further hydrocarbons other than ethyne. For the removal of soot, all of the devices employed for this purpose in known methods for the preparation of ethyne can be used, for example cyclones, scrubbers or electrostatic precipitators. For example, suitable devices are known from Ullmann's Encyclopedia of Industrial Chemistry, 5th Edition, Vol. A1, pages 108-110 and 118.

The plant for the electrothermic preparation of ethyne preferably comprises, in addition to the apparatus for separating soot, an apparatus for separating ethyne from the product gas obtained in the removal of soot. In the separation of ethyne, a gas containing hydrogen is obtained, which apart from hydrogen may contain fractions of ethyne which has not been separated and hydrocarbons other than ethyne. The apparatus for separating ethyne preferably comprises a compressor, an absorption column operated under pressure and a desorption column operated at a pressure lower than that in the absorption column. Water or suitable solvents, such as, for example, N-methylpyrrolidone, dimethylformamide or methanol, can be used for the selective absorption of ethyne. Suitable apparatuses for separating ethyne are known from the prior art, for example from Ullmann's Encyclopedia of Industrial Chemistry, 5th Edition, Vol. A1, pages 110-112.

The apparatus of the invention comprises, in addition to the plant for the electrothermic preparation of ethyne, a plant for the production of iron by reduction of oxidic iron ores. The plant for the production of iron by reduction of oxidic iron ores can be a blast furnace or a plant for direct reduction of oxidic iron ores. Suitable plants for the direct reduction of oxidic iron ores are known from Ullmann's Encyclopedia of Industrial Chemistry, Online addition 2012, keyword Iron, 3. Direct Reduction Processes, DOI 10.1002/14356007.o14_o02.

The plant for the production of iron by reduction of oxidic iron ores is preferably a blast furnace. In a blast furnace, oxidic iron ores are reduced predominantly by carbon monoxide which is generated in the blast furnace by air oxidation of coke.

The apparatus of the invention comprises, in addition to the plant for the electrothermic preparation of ethyne and the plant for the production of iron by reduction of oxidic iron ores, at least one gas conduit which feeds the hydrogen-containing gas from the plant for the electrothermic preparation of ethyne as reducing agent to the plant for the production of iron by reduction of oxidic iron ores. The apparatus of the invention can also comprise a plurality of gas conduits which each feed hydrogen-containing gas of differing composition from the plant for the electrothermic preparation of ethyne as reducing agent to the plant for the production of iron by reduction of oxidic iron ores. A conveyor device, e.g. a blower, can additionally be arranged in the gas conduit in order to transport the hydrogen-containing gas from the plant for the electrothermic preparation of ethyne into the plant for the production of iron by reduction of oxidic iron ores.

In a preferred embodiment, the plant for the production of iron by reduction of oxidic iron ores is a blast furnace and the gas conduit or conduits feed the hydrogen-containing gas or the hydrogen-containing gases into a lower section of the blast furnace. The gas conduit preferably feeds the hydrogen-containing gas into the blast furnace at a point above the air inlets.

In a preferred embodiment, the apparatus of the invention comprises a gas conduit which allows feeding all or part of the reaction mixture obtained in the electrothermic preparation of ethyne as reducing agent directly to the plant for the production of iron by reduction of oxidic iron ores. Here, directly means that no components are separated from the reaction mixture. The reaction mixture is particularly preferably fed uncooled to the plant for iron production in order to introduce the heat energy of the reaction mixture as completely as possible into the plant for iron production. For this purpose, the gas conduit is preferably thermally insulated in this embodiment.

In another preferred embodiment of the apparatus of the invention, the plant for the electrothermic preparation of ethyne comprises an apparatus for separating soot from the reaction mixture obtained in the electrothermic preparation of ethyne to give a product gas, and also a gas conduit which allows feeding this product gas as reducing agent to the plant for the production of iron by reduction of oxidic iron ores.

In another preferred embodiment of the apparatus of the invention, the plant for the electrothermic preparation of ethyne comprises, in addition to an apparatus for separating soot from the reaction mixture obtained in the electrothermic preparation of ethyne providing a product gas, an apparatus for separating ethyne from this product gas to give a hydrogen-containing gas, and also a gas conduit which allows feeding this hydrogen-containing gas as reducing agent to the plant for the production of iron by reduction of oxidic iron ores. Separation of ethyne particularly preferably provides a gas which apart from hydrogen contains hydrocarbons other than ethyne.

The gas conduits of the above-described three preferred embodiments of the apparatus of the invention can also be present in combination with one another and feed hydrogen-containing gas of differing composition from the plant for the electrothermic preparation of ethyne as reducing agent to the plant for the production of iron by reduction of oxidic iron ores.

In the three above-described preferred embodiments, the gas conduit is preferably connected to an apparatus for the storage of gas, particularly preferably to a gasometer. The connection of the gas conduit to an apparatus for the storage of gas allows for constant feeding of hydrogen-containing gas to the plant for iron production even when the plant for the electrothermic preparation of ethyne is operated with a throughput varying over time.

The apparatus of the invention can additionally comprise a storage device for ethyne. This storage device allows for ongoing continuous operation of downstream reactions of ethyne to form further products even when the plant for the electrothermic preparation of ethyne is operated with a throughput varying over time. The storage of ethyne is preferably effected as a solution in a solvent, particularly preferably in a solvent which is used in an apparatus for separating ethyne for the absorption of ethyne from the product gas obtained in the removal of soot.

The apparatus of the invention can be connected to a weather forecast unit. Such a connection to a weather forecast unit makes it possible to adapt the operation of the apparatus so that, on the one hand, the plant for the electrothermic preparation of ethyne can be operated with a throughput that is altered over time according to the power available from wind energy and solar energy and, on the other hand, there is always sufficient ethyne available for the continuous operation of a downstream ethyne-consuming plant. Thus, depending on the result of the weather forecast, it is possible, for example, to bring a storage device for ethyne to a high fill level or low fill level. In addition, a plant for further processing of the ethyne can be set up and adjusted for altered operating modes.

The apparatus of the invention can also be operated coupled with ethyne-consuming plants. When hydrogen- or hydrocarbon-containing gases are obtained as by-product in the ethyne-consuming plants, the apparatus of the invention is preferably coupled to the ethyne-consuming plant in such a way that hydrogen- or hydrocarbon-containing gas obtained as by-product is fed into a gas conduit which passes hydrogen-containing gas from the plant for the electrothermic preparation of ethyne as reducing agent to the plant for production of iron by the reduction of oxidic iron ores.

The process of the invention for utilizing electric energy for the production of iron from oxidic iron ores comprises an electrothermic preparation of ethyne from coal or a hydrocarbon-containing gas providing a hydrogen-containing gas and introduction of this gas as reducing agent into a plant for the production of iron by reduction of oxidic iron ores. The process of the invention is preferably carried out in the apparatus of the invention described above. Preference is given to using a blast furnace as plant for the production of iron by reduction of oxidic iron ores.

In the process of the invention, the electrothermic preparation of ethyne preferably proceeds from a hydrocarbon-containing gas, particularly preferably from natural gas. In another preferred embodiment, the electrothermic preparation of ethyne proceeds from coal. Suitable processes for the electrothermic preparation of ethyne from a hydrocarbon-containing gas or from coal are known from the prior art, for example from Ullmann's Encyclopedia of Industrial Chemistry, 5th Edition, Vol. A1, pages 115-122, from DE 1 900 644 A1, from EP 0 133 982 A2 and from H. Brachold et al., Chem.-Ing.-Tech. 65 (1993), pages 293-297.

In the embodiment of the process of the invention in which the electrothermic preparation of ethyne proceeds from coal and the plant for the production of iron by reduction of oxidic iron ores is a blast furnace, the coke obtained as by-product in the electrothermic preparation of ethyne from coal is preferably separated from the reaction mixture obtained in the electrothermic preparation of ethyne and fed from above into the blast furnace. For this purpose, the separated coke can be granulated, pelletized or briquetted before being introduced into the blast furnace.

The process of the invention is preferably operated in such a way that the hydrogen-containing gas additionally contains hydrocarbons other than ethyne. These hydrocarbons can be unreacted components of a hydrocarbon-containing gas feed or can be formed as by-product in the electrothermic preparation of ethyne. As an alternative or in addition, hydrocarbons other than ethyne can be produced by mixing the reaction mixture obtained in the electrothermic preparation of ethyne with a hydrocarbon-containing gas or a hydrocarbon-containing liquid so that at least part of the added hydrocarbons is endothermically cracked.

In a preferred embodiment of the process of the invention, soot is separated from the reaction mixture obtained in the electrothermic preparation of ethyne and separated soot is fed as reducing agent to the plant for the production of iron by reduction of oxidic iron ores. The plant for the production of iron by reduction of oxidic iron ores is preferably a blast furnace and the soot is either fed as reducing agent into a lower section of the blast furnace or is fed together with coke from above into the blast furnace. To feed the soot together with coke into the blast furnace, the soot is preferably granulated, pelletized or briquetted before being introduced into the blast furnace.

In a preferred embodiment of the process of the invention, soot is first separated from the reaction mixture obtained in the electrothermic preparation of ethyne and ethyne is subsequently separated from the product gas obtained to provide a hydrogen-containing gas. In the separation of ethyne, the hydrocarbons other than ethyne present in the product gas are preferably not separated or only partly separated, so that the hydrogen-containing gas additionally contains all or part of the hydrocarbons other than ethyne present in the product gas. Such a selective isolation of ethyne makes it possible to utilize the hydrocarbons other than ethyne as reducing agent and for the generation of energy in the plant for the production of iron by reduction of oxidic iron ores and requires less energy and outlay in terms of apparatus than a complete separation of the product gas.

In the process of the invention, the throughput in the plant for the electrothermic preparation of ethyne is preferably altered as a function of the availability of electric energy. For this purpose the plant for the electrothermic preparation of ethyne can be switched on or off as desired, for example as a function of the current price for power on a power exchange. As an alternative, the plant for the electrothermic preparation of ethyne can also be operated with variable load so that its power consumption corresponds to a current excess of electric energy. The plant for the electrothermic preparation of ethyne is preferably operated using excess electric energy. Excess electric energy can originate from a power generator located next to the apparatus of the invention, for example from a neighbouring power plant, a neighbouring wind generator or a neighbouring photovoltaic plant. Excess electric energy is particularly preferably drawn from a power grid. Excess electric energy can in this case be drawn as negative regulating energy from a power grid in order to compensate for an excess of power input into the grid relative to the current power offtake. The excess electric energy used for the process of the invention is preferably energy generated from wind energy or solar energy.

The amount of the gas which is obtained in the electrothermic preparation of ethyne and is fed as reducing agent to the plant for the production of iron by reduction of oxidic iron ores is preferably also altered as a function of the availability of electric energy. The amount of the gas fed to the plant for iron production can be altered to the same extent as the throughput in the plant for the electrothermic preparation of ethyne. The amount of the gas fed to the plant for iron production can, however, also be altered to a lesser extent than the throughput in the plant for the electrothermic preparation of ethyne. For example, when the throughput in the plant for the electrothermic preparation of ethyne is low, the entire amount of hydrogen produced in this plant can be fed as reducing agent to the plant for the production of iron by reduction of oxidic iron ores and when the throughput in the plant for the electrothermic preparation of ethyne is high, only part of the hydrogen produced can be fed to the plant for iron production and the other part can be fed to a storage device or another use.

As an alternative to or in combination with the embodiment described in the preceding paragraph, the composition of the gas which is obtained in the electrothermic preparation of ethyne and is fed as reducing agent to the plant for the production of iron by reduction of oxidic iron ores can be altered as a function of the availability of electric energy. For example, the process can be carried out in an apparatus which comprises both a gas conduit which feeds an ethyne- and hydrogen-containing gas, before ethyne has been separated, to the plant for iron production and also a gas conduit which, after ethyne has been separated, feeds an ethyne-depleted, hydrogen-containing gas to the plant for iron production. The apparatus can be operated in such a way that when the throughput in the plant for the electrothermic preparation of ethyne is low, predominantly ethyne- and hydrogen-containing gas is fed to the plant for iron production and when the throughput in the plant for the electrothermic preparation of ethyne is high, predominantly an ethyne-depleted, hydrogen-containing gas is fed to the plant for iron production so that the fluctuation in the amount of reducing agent fed to the plant for the production of iron by reduction of oxidic iron ores is kept low. However, the apparatus can also be operated in such a way that when the throughput in the plant for the electrothermic preparation of ethyne is low, predominantly an ethyne-depleted, hydrogen-containing gas is fed to the plant for iron production and when the throughput in the plant for the electrothermic preparation of ethyne is high, predominantly an ethyne- and hydrogen-containing gas is fed to the plant for iron production, so that the capacity of the apparatus for separating ethyne from the product gas can be kept small.

As an alternative to or in combination with the embodiments described in the two preceding paragraphs, the temperature of the gas which is obtained in the electrothermic preparation of ethyne and is fed as reducing agent to the plant for the production of iron by reduction of oxidic iron ores can be altered as a function of the availability of electric energy. For example, the process can be carried out in an apparatus which comprises both a first gas conduit which can feed all or part of the reaction mixture obtained in the electrothermic preparation of ethyne directly as reducing agent to the plant for the production of iron by reduction of oxidic iron ores and also a second gas conduit which feeds a hydrogen-containing gas obtained after cooling of the reaction mixture to the plant for iron production. The apparatus can be operated in such a way that when the throughput in the plant for the electrothermic preparation of ethyne is low, predominantly reaction mixture obtained in the electrothermic preparation of ethyne is fed directly without cooling through the first gas conduit to the plant for iron production and when the throughput in the plant for the electrothermic preparation of ethyne is high, predominantly a hydrogen-containing gas obtained after cooling of the reaction mixture is fed through the second gas conduit to the plant for iron production so that the fluctuation in the amount of heat introduced into the plant for the production of iron by reduction of oxidic iron ores is kept low. However, the apparatus can also be operated in such a way that when the throughput in the plant for the electrothermic preparation of ethyne is low, predominantly a hydrogen-containing gas obtained after cooling of the reaction mixture is fed through the second gas conduit to the plant for iron production and when the throughput in the plant for the electrothermic preparation of ethyne is high, predominantly a reaction mixture obtained in the electrothermic preparation of ethyne is fed directly without cooling through the first gas conduit to the plant for iron production so that the capacity of the apparatus for cooling the reaction mixture from the electrothermic preparation of ethyne can be kept small.

In a preferred embodiment, the process of the invention is carried out in an apparatus which additionally comprises a gas storage device. When the availability of electric energy is high, the plant for the electrothermic preparation of ethyne is operated at a higher conversion and gas obtained in the electrothermic preparation of ethyne is fed to a gas storage device. When the availability of electric energy is low, the plant for the electrothermic preparation of ethyne is operated at a lower conversion and gas is withdrawn from the gas storage device. The amount of the gas, obtained in the electrothermic preparation of ethyne, which is fed as reducing agent to the plant for the production of iron by reduction of oxidic iron ores is particularly preferably kept essentially constant. In this embodiment, the throughput in the plant for the electrothermic preparation of ethyne can change quickly and within wide limits as a function of the availability of electric energy without there being adverse effects on the operation of the plant for the production of iron by reduction of oxidic iron ores.

Compared to a blast furnace process, the process of the invention allows the CO₂ emission in the production of iron from oxidic iron ores to be significantly reduced. In the embodiments of the process in which ethyne is separated from the reaction mixture obtained in the electrothermic preparation of ethyne and is not fed to the plant for the production of iron by reduction of oxidic iron ores, the CO₂ emissions from the plant for reduction of oxidic iron ores are reduced. In the embodiments of the process in which the ethyne produced is also fed to the plant for the production of iron by reduction of oxidic iron ores, part of the energy requirement for the reduction of the oxidic iron ores is covered by the higher energy content of ethyne compared to the starting materials and optionally by the heat energy which can be introduced with the reaction mixture obtained in the electrothermic preparation of ethyne into the plant for iron production, so that, for example, in the case of a blast furnace the amount of air introduced and the fraction of coke which has to be burnt for heat generation in the blast furnace are reduced. This also contributes to a reduction in the CO₂ emissions.

Compared to production of hydrogen by electrolysis of water, the process of the invention has the advantage that in the electrothermic preparation of ethyne less electric energy is required for the production of the same amount of hydrogen and the heat energy obtained can also be utilized, for example for steam generation in the plant for the electrothermic preparation of ethyne or for heating the plant for iron production by feeding the reaction mixture obtained in the electrothermic preparation of ethyne directly without cooling to the plant for iron production, so that the electric energy introduced is utilized more efficiently.

1-21. (canceled)

22. An apparatus for utilizing electric energy for producing iron from oxidic iron ores, comprising:
a) a plant for an electrothermic preparation of ethyne from a hydrocarbon-containing gas, which plant provides a hydrogen-containing gas;
b) a plant for the production of iron by reduction of oxidic iron ores; and
c) at least one gas conduit which feeds the hydrogen-containing gas from the plant for the electrothermic preparation of ethyne as reducing agent to the plant for the production of iron by reduction of oxidic iron ores.

23. The apparatus of claim 22, wherein the plant for the production of iron by reduction of oxidic iron ores is a blast furnace and the gas conduit feeds the hydrogen-containing gas into a lower section of the blast furnace.

24. The apparatus of claim 22, wherein the plant for the production of iron by reduction of oxidic iron ores is a plant for direct reduction of oxidic iron ores.

25. The apparatus of claim 22, wherein the plant for the electrothermic preparation of ethyne comprises an electric arc reactor.

26. The apparatus of claim 22, comprising a gas conduit which allows feeding all or part of the reaction mixture obtained in the electrothermic preparation of ethyne as reducing agent directly to the plant for the production of iron by reduction of oxidic iron ores.

27. The apparatus of claim 26, wherein the gas conduit, which allows feeding all or part of the reaction mixture, is thermally insulated.

28. The apparatus of claim 22, wherein the plant for the electrothermic preparation of ethyne comprises an apparatus for separating soot from the reaction mixture obtained in the electrothermic preparation of ethyne providing a product gas and the apparatus comprises a gas conduit which allows feeding this product gas as reducing agent to the plant for the production of iron by reduction of oxidic iron ores.

29. The apparatus of claim 22, wherein the plant for the electrothermic preparation of ethyne comprises an apparatus for separating soot from the reaction mixture obtained in the electrothermic preparation of ethyne providing a product gas and an apparatus for separating ethyne from this product gas providing a hydrogen-containing gas and the apparatus comprises a gas conduit which allows feeding this hydrogen-containing gas as reducing agent to the plant for the production of iron by reduction of oxidic iron ores.

30. The apparatus of claim 29, further comprising an apparatus for separating ethyne from the product gas providing a gas containing hydrogen and hydrocarbons other than ethyne.

31. The apparatus of claim 28, wherein the gas conduit, which allows feeding said product gas, is connected to a device for storing gas.

32. A process for utilizing electric energy for producing iron from oxidic iron ores, comprising an electrothermic preparation of ethyne from a hydrocarbon-containing gas providing a hydrogen-containing gas, and feeding this gas as reducing agent into a plant for the production of iron by reduction of oxidic iron ores.

33. The process of claim 32, carried out in an apparatus comprising:
a) a plant for an electrothermic preparation of ethyne from a hydrocarbon-containing gas, which plant provides a hydrogen-containing gas;
b) a plant for the production of iron by reduction of oxidic iron ores; and
c) at least one gas conduit which feeds the hydrogen-containing gas from the plant for the electrothermic preparation of ethyne as reducing agent to the plant for the production of iron by reduction of oxidic iron ores.

34. The process of claim 32, wherein the electrothermic preparation of ethyne proceeds from natural gas.

35. The process of claim 32, wherein the hydrogen-containing gas additionally contains hydrocarbons other than ethyne.

36. The process of claim 32, wherein soot is separated from the reaction mixture obtained in the electrothermic preparation of ethyne and separated soot is fed as reducing agent to the plant for the production of iron by reduction of oxidic iron ores.

37. The process of claim 32, wherein the throughput in the plant for the electrothermic preparation of ethyne is altered as a function of the availability of electric energy.

38. The process of claim 37, wherein the amount of the gas which is obtained in the electrothermic preparation of ethyne and is fed as reducing agent to the plant for the production of iron by reduction of oxidic iron ores is altered as a function of the availability of electric energy.

39. The process of claim 37, wherein the composition of the gas which is obtained in the electrothermic preparation of ethyne and is fed as reducing agent to the plant for the production of iron by reduction of oxidic iron ores is altered as a function of the availability of electric energy.

40. The process of claim 37, wherein the temperature of the gas obtained in the electrothermic preparation of ethyne which is fed as reducing agent to the plant for the production of iron by reduction of oxidic iron ores is altered as a function of the availability of electric energy.

41. The process of claim 37, wherein, when availability of electric energy is high, the plant for the electrothermic preparation of ethyne is operated at a higher conversion and gas obtained in the electrothermic preparation of ethyne is fed to a gas storage device, and when availability of electric energy is low, the plant for the electrothermic preparation of ethyne is operated at a lower conversion and gas is taken from the gas storage device.

42. The process of claim 41, wherein the amount of the gas obtained in the electrothermic preparation of ethyne which is fed as reducing agent to the plant for the production of iron by reduction of oxidic iron ores is kept essentially constant.
